(19) United States
(12) Patent Application Publication
(10) Pub. No.: US 2010/0227889 A1
(43) Pub. Date: Sep. 9, 2010

(54) Benzimidazole Derivatives

(76) Inventors: Marc Gerspacher, Kappel (CH); Karl Heinz Krawinkler, Basel (CH)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/295,381

(22) PCT Filed: Mar. 28, 2007
(86) PCT No.: PCT/EP07/02763

§ 371 Date: Sep. 30, 2008

(30) Foreign Application Priority Data
Mar. 30, 2006 (GB) 0606426.5

(51) Int. Cl. A61K 31/4439; C07D 401/10; A61P 19/08

(52) U.S. Cl. 514/338; 546/273.4

(57) ABSTRACT

A compound of formula (I) or a pharmaceutically acceptable salt or prodrug ester thereof: wherein R, X and Y are as disclosed in the specification, suitable for the treatment of osteoporosis.

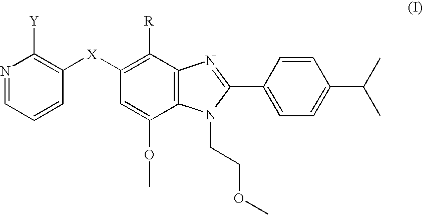

The present invention relates to bicyclic compounds, in particular to benzimidazole derivatives and to pharmaceutical uses thereof.

Accordingly the invention provides compounds of formula (I) or a pharmaceutically acceptable salt or prodrug ester thereof:

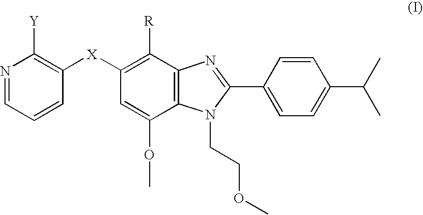

wherein
R is halo or optionally substituted C₁-C₆ alkyl;
X is selected from the group consisting of O, NH, CH₂, CO, SO, SO₂ or S;
Y represents a group selected from the following: optionally substituted C₁-C₆ alkyl, —SR₁, —S(O)R₁, —S(O)₂R₁, —OR₂, wherein R₁ and R₂ are selected from optionally substituted: C₁-C₄ alkyl, C₁-C₄ alkenyl or C₁-C₄ alkynyl;
the optional substituent or substituents on R, R₁, R₂ and Y being independently selected from the group consisting of halogen, hydroxy, C₁-C₆ alkyl, mono or di-C₁-C₆ alkylamino, aminocarbonyl, sulfinyl, sulfonyl, sulfanyl, mono or di-C₁-C₆ alkylaminocarbonyl, amino, carboxy, C₁-C₆ alkoxy, C₂-C₆ alkenyloxy, C₂-C₆ alkynyloxy, C₃-C₁₂ cycloalkyl, C₃-C₁₈ heterocycloalkyl, C₁-C₆ alkylcarbonyl, C₁-C₆ alkoxycarbonyl, nitryl, aryl; all of which, except halogen, are independently optionally substituted by one or more substituents, selected from the group consisting of halogen, hydroxy, C₁-C₆ alkyl, mono or di-C₁-C₆ alkylamino, aminocarbonyl, sulfinyl, sulfonyl, sulfanyl, mono or di-C₁-C₆ alkylaminocarbonyl, amino, carboxy, C₁-C₈ alkoxy, C₃-C₁₂ cycloalkyl, C₃-C₁₈ heterocycloalkyl, C₁-C₈ alkylcarbonyl, C₁-C₈ alkoxycarbonyl, nitryl, aryl.

Additionally the invention provides compounds of formula (I) or a pharmaceutically acceptable salt or prodrug ester thereof:

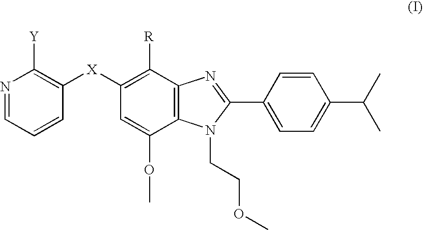

wherein
R is halo or optionally substituted C₁-C₈ alkyl;
X is selected from the group consisting of O, NH, CH₂, CO, SO, SO₂ or S;
Y represents a group selected from the following: optionally substituted C₁-C₈ alkyl, —SR₁, —S(O)R₁, —S(O)₂R₁, —OR₁, wherein R₁ is C₁-C₄ alkyl;
the optional substituent or substituents on R and Y being independently selected from the group consisting of halogen, hydroxy, C₁-C₈ alkyl, mono or di-C₁-C₈ alkylamino, aminocarbonyl, sulfinyl, sulfonyl, sulfanyl, mono or di-C₁-C₈ alkylaminocarbonyl, amino, carboxy, C₁-C₆ alkoxy, C₃-C₁₂ cycloalkyl, C₃-C₁₈ heterocycloalkyl, C₁-C₈ alkylcarbonyl, C₁-C₈ alkoxycarbonyl, nitryl, aryl; all of which, except halogen, are independently optionally substituted by one or more substituents, selected from the group consisting of halogen, hydroxy, C₁-C₈ alkyl, mono or di-C₁-C₈ alkylamino, aminocarbonyl, sulfinyl, sulfonyl, sulfanyl, mono or di-C₁-C₈ alkylaminocarbonyl, amino, carboxy, C₁-C₈ alkoxy, C₃-C₁₂ cycloalkyl, C₃-C₁₈ heterocycloalkyl, C₁-C₈ alkylcarbonyl, C₁-C₈ alkoxycarbonyl, nitryl, aryl.

For the avoidance of doubt, the terms listed below are to be understood to have the following meaning throughout the present description and claims:

The term “lower”, when referring to organic radicals or compounds means a compound or radical with may be branched or unbranched with up to and including 7 carbon atoms.

A lower alkyl group may be branched, unbranched or cyclic and contains 1 to 7 carbon atoms, preferably 1 to 4 carbon atoms. Lower alkyl represents, for example: methyl, ethyl, propyl, butyl, isopropyl, isobutyl, tertiary butyl or 2,2-dimethylpropyl.

A lower alkoxy group may be branched or unbranched and contains 1 to 7 carbon atoms, preferably 1 to 6 carbon atoms. Lower alkoxy represents, for example: methoxy, ethoxy, propoxy, butoxy, isopropoxy, isobutoxy or tertiary butoxy. Lower alkoxy includes cycloalkyloxy and cycloalkyl-lower alkyloxy.

A lower alkene, alkenyl or alkenoxy group is branched or unbranched and contains 2 to 7 carbon atoms, preferably 1 to 4 carbon atoms and contains at least one carbon-carbon double bond. Lower alkene, lower alkenyl or lower alkenyloxy represents for example vinyl, prop-1-enyl, allyl, butenyl, isopropenyl or isobutenyl and the oxy equivalents thereof.

A lower akyne or alkynyl group is branched or unbranched and contains 2 to 7 carbon atoms, preferably 1 to 4 carbon atoms and contains at least one carbon-carbon triple bond. Lower alkyne or lower alkynyl or lower alkenyloxy represents for example ethynyl, propynyl or propargyl.

In the present application, oxygen containing substituents, e.g. alkoxy, alkenyloxy, alkynyloxy, carbonyl, etc. encompass their sulphur containing homologues, e.g. thioalkyl, alkyl-thioalkyl, thioalkenyl, alkenyl-thioalkyl, thioalkynyl, thiocarbonyl, sulphone, sulphoxide etc.

Halo or halogen represents chloro, fluoro, bromo or iodo.

Aryl represents carbocyclic aryl, heterocyclic aryl or biaryl.

Carbocyclic aryl is an aromatic cyclic hydrocarbon containing from 6 to 18 ring atoms. It can be monocyclic, bicyclic or tricyclic, for example naphthyl, phenyl, or phenyl mono-, di- or trisubstituted by one, two or three substituents.

Heterocyclic aryl is an aromatic monocyclic or bicyclic hydrocarbon containing from 5 to 18 ring atoms one or more of which are heteroatoms selected from O, N or S. Preferably there are one or two heteroatoms. Heterocyclic aryl represents, for example: pyridyl, indolyl, quinoxalinyl, quinolinyl, isoquinolinyl, benzothienyl, benzofuranyl, benzopyranyl, benzothiopyranyl, furanyl, pyrrolyl, thiazolyl, oxazolyl, isoxazolyl, triazolyl, tetrazolyl, pyrazolyl, imidazolyl, thienyl, oxadiazolyl, benzimidazolyl. Heterocyclic aryl also includes such substituted radicals.

Cycloalkyl represents a cyclic hydrocarbon containing from 3 to 12 ring atoms preferably from 3 to 6 ring atoms. Cycloalkyl represents, for example: cyclopropyl, cyclobutyl, cyclopentyl, or cyclohexyl. The cycloalkyl may optionally be substituted.

Heterocycloalkyl represents a mono-, di- or tricyclic hydrocarbon which may be saturated or unsaturated and which contains one or more, preferably one to three heteroatoms selected from O, N or S. Preferably it contains between three and 18 ring atoms. The term heterocycloalkyl is intended also to include bridged heterocycloalkyl groups such as 3-hyroxy-8-aza-bicyclo[3.2.1]oct-8-yl.

Pharmaceutically acceptable salts include acid addition salts with conventional acids, for example mineral acids, e.g. hydrochloric acid, sulfuric or phosphoric acid, or organic acids, for example aliphatic or aromatic carboxylic or sulfonic acids, e.g. acetic, trifluoroacetic, propionic, succinic, glycolic, lactic, malic, tartaric, citric, ascorbic, maleic, fumaric, hydroxylmaleic, pyruvic, pamoic, methanesulfonic, toluenesulfonic, naphthalenesulfonic, sulfanilic or cyclohexylsulfamic acid; also amino acids, such as arginine and lysine. For compounds of the invention having acidic groups, for example a free carboxy group, pharmaceutically acceptable salts also represent metal or ammonium salts, such as alkali metal or alkaline earth metal salts, e.g. sodium, potassium, magnesium or calcium salts, as well as ammonium salts, which are formed with ammonia or suitable organic amines.

The agents of the invention which comprise free hydroxyl groups may also exist in the form of pharmaceutically acceptable, physiologically cleavable esters, and as such are included within the scope of the invention. Such pharmaceutically acceptable esters are preferably prodrug ester derivatives, such being convertible by solvolysis or cleavage under physiological conditions to the corresponding agents of the invention which comprise free hydroxyl groups. Suitable pharmaceutically acceptable prodrug esters are those derived from a carboxylic acid, a carbonic acid monoester or a carbamic acid, advantageously esters derived from an optionally substituted lower alkanoic acid or an arylcarboxylic acid.

In preferred compounds of formula (I), X is CH₂ or O.

More preferably, X is CH₂.

A second aspect of the invention provides a compound of formula (I′) or a pharmaceutically acceptable salt, or prodrug ester thereof:

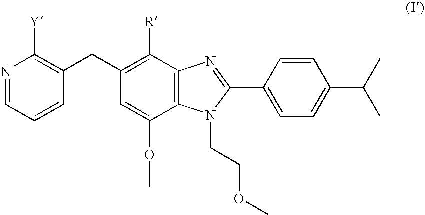

wherein
R′ is halo or optionally substituted C₁-C₆ alkyl;
Y′ represents a group selected from the following: C₁-C₆ alkyl, —SR₁, —S(O)R₁, —S(O)₂R₁, —OR₂, wherein R₁ and R₂ are selected from optionally substituted: C₁-C₄ alkyl, C₂-C₄ alkenyl or C₂-C₄ alkynyl;
the optional substituent or substituents on R, R₁ and R₂ are independently selected from the group consisting of halogen, hydroxy, C₁-C₆ alkyl, mono or di-C₁-C₆ alkylamino, aminocarbonyl, sulfinyl, sulfonyl, sulfanyl, mono or di-C₁-C₆ alkylaminocarbonyl, amino, carboxy, C₁-C₆ alkoxy, C₃-C₁₂ cycloalkyl, C₃-C₁₈ heterocycloalkyl, C₁-C₆ alkylcarbonyl, C₁-C₆ alkoxycarbonyl, nitryl, aryl; all of which, except halogen, are independently optionally substituted by one or more substituents, selected from the group consisting of halogen, hydroxy, C₁-C₆ alkyl, mono or di-C₁-C₆ alkylamino, aminocarbonyl, sulfinyl, sulfonyl, sulfanyl, mono or di-C₁-C₆ r alkylaminocarbonyl, amino, carboxy, C₁-C₆ alkoxy, C₃-C₁₂ cycloalkyl, C₃-C₁₈ heterocycloalkyl, C₁-C₆ alkylcarbonyl, C₁-C₆ alkoxycarbonyl, nitryl, aryl.

Additionally in second aspect, the invention provides a compound of formula (I′) or a pharmaceutically acceptable salt, or prodrug ester thereof:

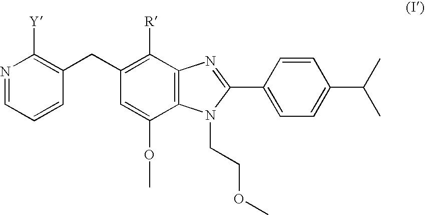

wherein
R′ is halo or optionally substituted C₁-C₆ alkyl;
Y′ represents a group selected from the following: C₁-C₆ alkyl, —SR₁, —S(O)R₁, —S(O)₂R₁, —OR₁, wherein R₁ is C₁-C₄ alkyl;

The optional substituent or substituents on R are independently selected from the group consisting of halogen, hydroxy, C₁-C₆ alkyl, mono or di-C₁-C₆ alkylamino, aminocarbonyl, sulfinyl, sulfonyl, sulfanyl, mono or di-C₁-C₆ alkylaminocarbonyl, amino, carboxy, lower alkoxy, C₃-C₁₂ cycloalkyl, C₃-C₁₈ heterocycloalkyl, C₁-C₆ alkylcarbonyl, C₁-C₆ alkoxycarbonyl, nitryl, aryl; all of which, except halogen, are independently optionally substituted by one or more substituents, selected from the group consisting of halogen, hydroxy, C₁-C₆ alkyl, mono or di-C₁-C₆ alkylamino, aminocarbonyl, sulfinyl, sulfonyl, sulfanyl, mono or di-C₁-C₆ alkylaminocarbonyl, amino, carboxy, C₁-C₆ alkoxy, C₃-C₁₂ cycloalkyl, C₃-C₁₈ heterocycloalkyl, C₁-C₆ alkylcarbonyl, C₁-C₆ alkoxycarbonyl, nitryl, aryl.

With respect to the above described compounds of formula (I) and formula (I′) one or more of the following significances may apply:

Preferably, Y is selected from: —OR₂, —S(O)R₁, and —S(O)₂R₁.

More preferably, Y is selected from —OR₂ and —SR₁, yet more preferably —OR₂.

Alternatively preferably, Y is selected from: —SR₁, —S(O)R₁ and —S(O)₂R₁.

R₁ is preferably optionally substituted C₁-C₄ alkyl or C₁-C₄ alkynyl.

R₁ is more preferably optionally substituted C₁-C₄ alkyl.

More preferably, R₁ or R₂ is methyl.

Yet more preferably, Y is selected from: —SMe, —S(O)Me and —S(O)₂Me.

Preferably R is halo or trifluoromethyl.

More preferably R is trifluoromethyl.

Preferred compounds of formula I are:
4-Bromo-2-(4-isopropyl-phenyl)-7-methoxy-1-(2-methoxy-ethyl)-5-(2-methylsulfanyl-pyridin-3-ylmethyl)-1H-benzoimidazole
2-(4-Isopropyl-phenyl)-7-methoxy-1-(2-methoxy-ethyl)-5-(2-methylsulfanyl-pyridin-3-ylmethyl)-4-trifluoromethyl-1H-benzoimidazole
4-Bromo-2-(4-isopropyl-phenyl)-5-(2-methanesulfinyl-pyridin-3-ylmethyl)-7-methoxy-1-(2-methoxy-ethyl)-1H-benzoimidazole
2-(4-Isopropyl-phenyl)-5-(2-methanesulfinyl-pyridin-3-ylmethyl)-7-methoxy-1-(2-methoxy-ethyl)-4-trifluoromethyl-1H-benzoimidazole
2-(4-Isopropyl-phenyl)-5-(2-methanesulfonyl-pyridin-3-ylmethyl)-7-methoxy-1-(2-methoxy-ethyl)-4-trifluoromethyl-1H-benzoimidazole
2-(4-Isopropyl-phenyl)-7-methoxy-1-(2-methoxy-ethyl)-5-(2-methoxy-pyridin-3-ylmethyl)-4-trifluoromethyl-1H-benzoimidazole
5-(2-Ethoxy-pyridin-3-ylmethyl)-2-(4-Isopropyl-phenyl)-7-methoxy-1-(2-methoxy-ethyl)-4-trifluoromethyl-1H-benzoimidazole
5-(2-Isopropoxy-pyridin-3-yl methyl)-2-(4-Isopropyl-phenyl)-7-methoxy-1-(2-methoxy-ethyl)-4-trifluoromethyl-1H-benzoimidazole
2-(4-Isopropyl-phenyl)-7-methoxy-1-(2-methoxy-ethyl)-5-(2-prop-2-ynyloxy-pyridin-3-ylmethyl)-4-trifluoromethyl-1H-benzoimidazole
2-(4-Isopropyl-phenyl)-7-methoxy-5-[2-(2-methoxy-ethoxy)-pyridin-3-ylmethyl]-1-(2-methoxy-ethyl)-4-trifluoromethyl-1H-benzoimidazole
(2-{3-[2-(4-Isopropyl-phenyl)-7-methoxy-1-(2-methoxy-ethyl)-4-trifluoromethyl-1H-benzoimidazole-5-ylmethyl]-pyridin-2-yloxy}-ethyl)-dimethylamine.

According to a third aspect of the invention there is provided a pharmaceutical composition comprising a compound of formula (I) in association with a pharmaceutically acceptable excipient, diluent or carrier.

According to a fourth aspect of the invention there is provided a compound of formula (I) for promoting the release of parathyroid hormone.

It is now well established that controlled treatment of patients with parathyroid hormone (PTH) and analogues and fragments thereof can have a pronounced anabolic effect on bone formation. Thus compounds which promote PTH release, such as the compounds of the present invention may be used for preventing or treating conditions of bone which are associated with increased calcium depletion or resorption or in which stimulation of bone formation and calcium fixation in the bone is desirable.

Thus in a fifth aspect the invention includes a method for preventing or treating bone conditions which are associated with increased calcium depletion or resorption or in which stimulation of bone formation and calcium fixation in the bone is desirable in which an effective amount of a compound of formula (I) as defined above, or a pharmaceutically-acceptable and -cleavable ester, or acid addition salt thereof is administered to a patient in need of such treatment.

In a sixth aspect the invention provides a process for preparation of a compound of formula (I) in free or salt form, comprising:

(a) for compounds of formula (I) wherein R is optionally substituted C₁-C₆ alkyl, introducing the optionally substituted C₁-C₆ alkyl by reaction of a compound of formula (XV) with a suitable organometallic reagent:

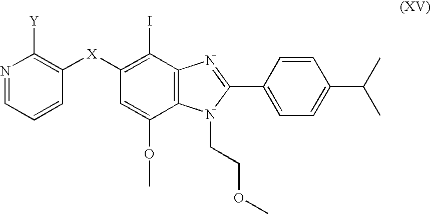

(b) for compounds of formula (I) wherein R is halo, halogenation of a compound of formula (X) using a suitable halogenating agent:

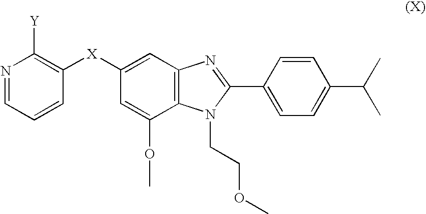

(c) for compounds of formula (I) wherein Y is —SR₁, reduction of a compound of formula (XI) using a suitable reducing agent:

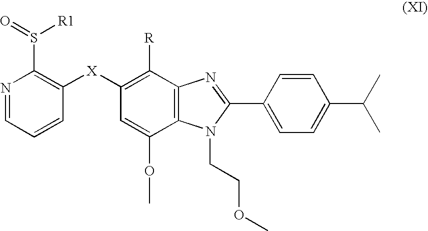

(d) for compounds wherein Y is —S(O)R₁ or —S(O)₂R₁, by oxidation of a compound of formula (XII):

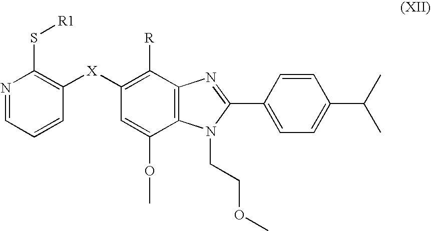

(e) for compounds wherein Y is —OR₂, or —SR₁ by ipso-substitution in the pyridine ring of a compound of formula (XIII):

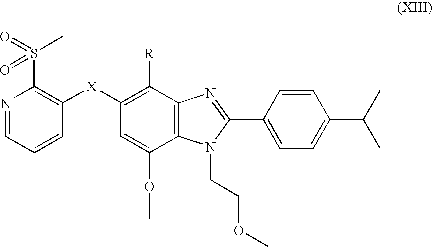

In step (a), an example of a suitable reagent for introduction of a methyl group at the R position would be Me₂CuLi.

In step (b), bromination, for example, of the compound of formula (XV) may be carried out using bromine/acetic acid.

In step (c), 4-toluene-suphonic acid, sodium iodide in acetonitrile may conveniently be used to effect the reduction of the compound (XI).

In step (d), oxidation can be conveniently carried out for example using hydrogen peroxide and acetic acid.

In step (e), selective Ipso-substitution in the pyridine ring can be achieved with nucleophiles such as R₂O⁻ and R₁S⁻.

The abovementioned compounds of formula (XV), (XI), (XII) and (XIII) may be prepared as outlined in the following schemes:

Synthesis of compounds according to the invention of formula (I) wherein X is —CH₂— is further illustrated by the following Scheme 1:

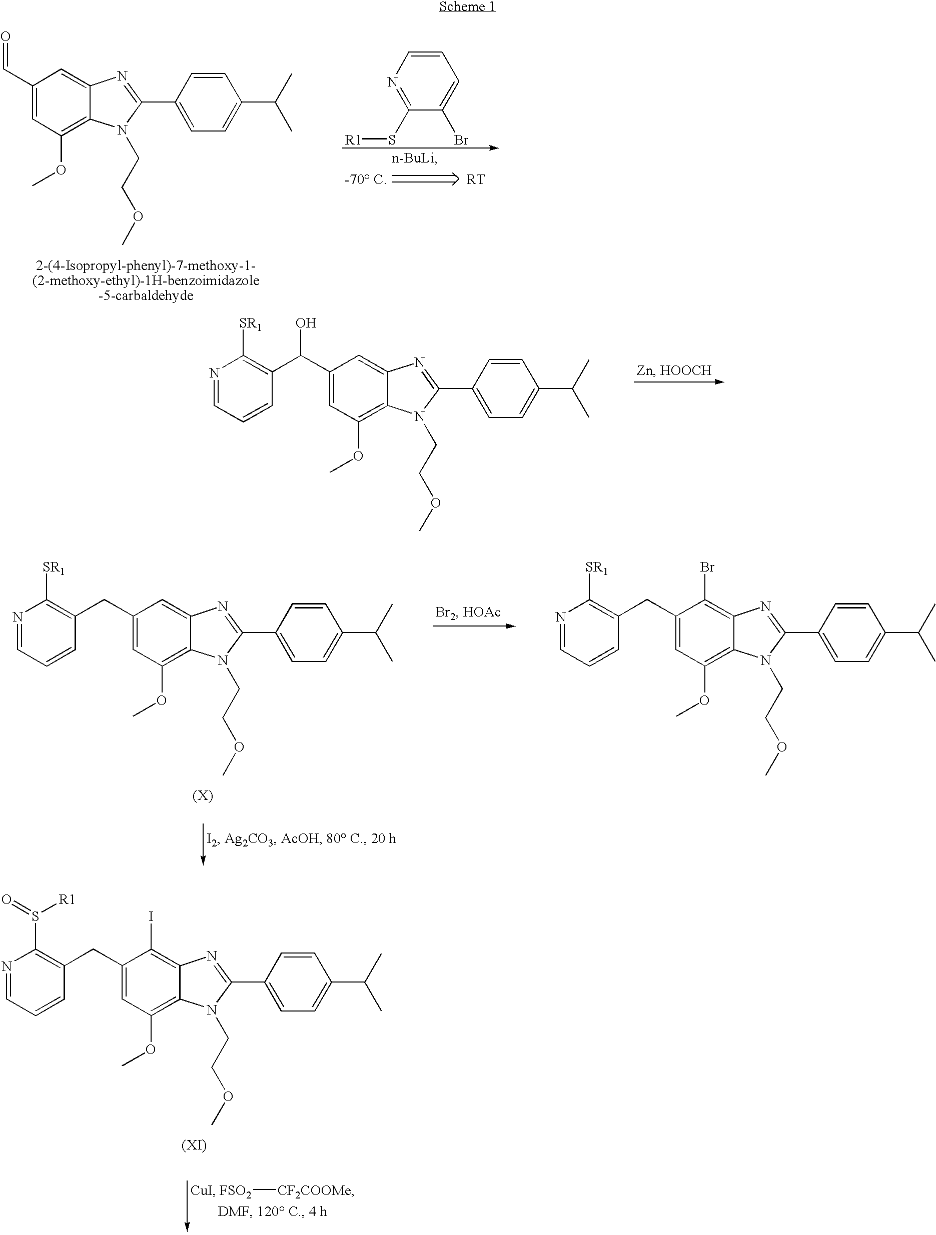

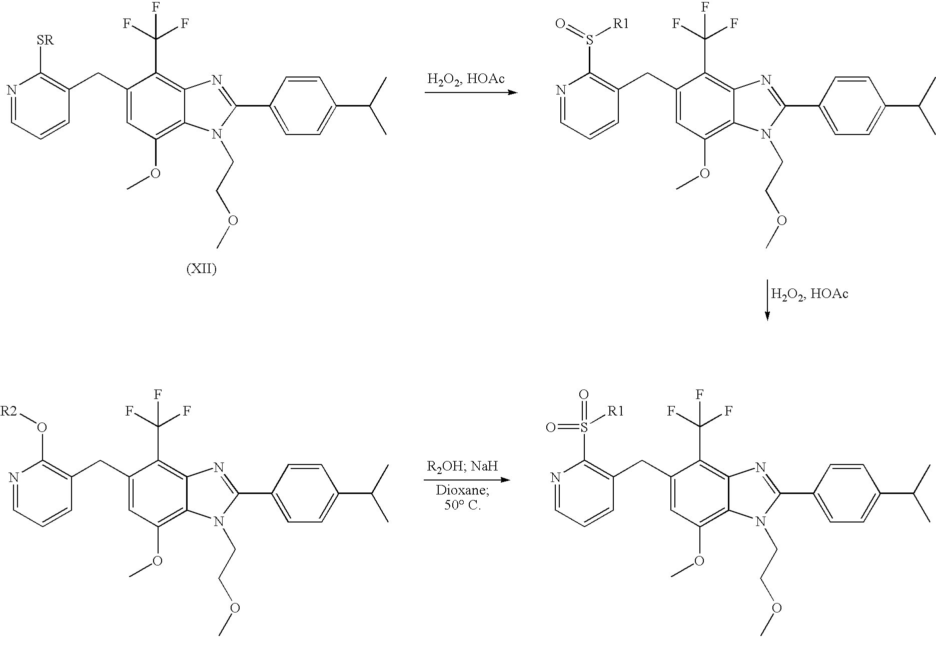

Compounds of the invention wherein X is a group other than —CH₂— may for example be prepared according to the following Scheme 2:

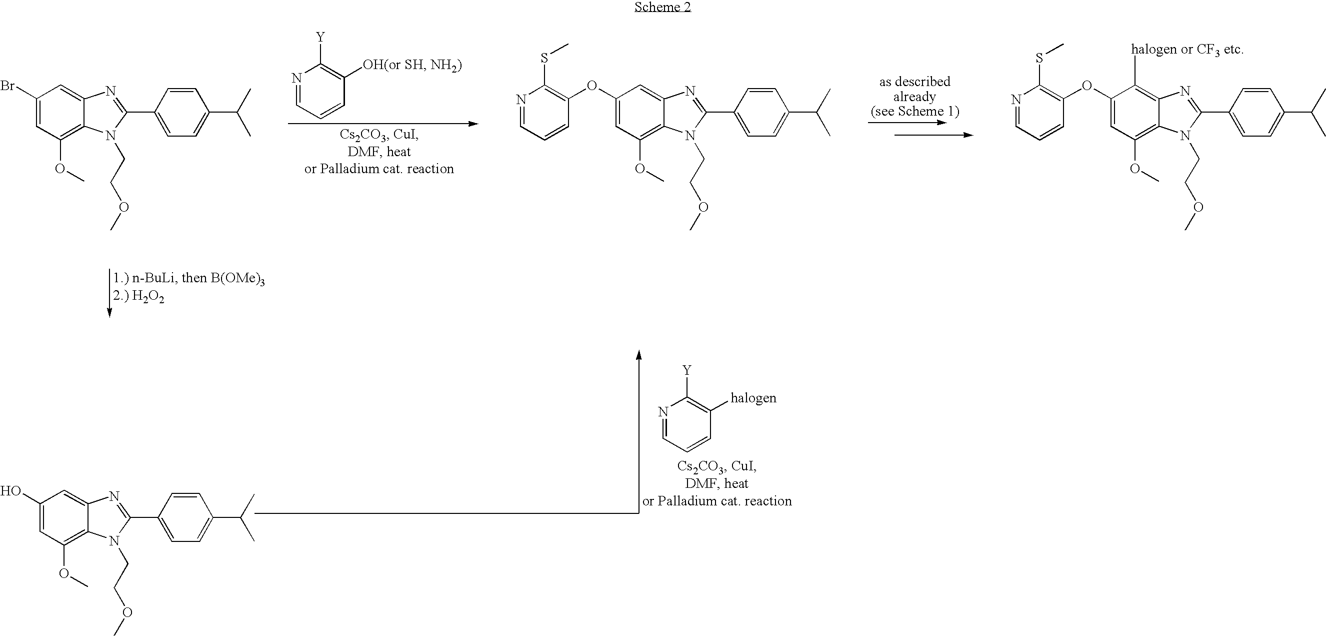

The compounds of formula I in free form may be converted into salt forms in conventional manner and vice-versa.

The compounds of the invention can be recovered from the reaction mixture and purified in conventional manner. Isomers, such as enantiomers, may be obtained in conventional manner, e.g. by fractional crystallization or asymmetric synthesis from corresponding asymmetrically substituted, e.g. optically active starting materials.

In a seventh aspect invention includes the use of a compound of formula (I) in the manufacture of a medicament for preventing or treating bone conditions which are associated with increased calcium depletion or resorption or in which stimulation of bone formation and calcium fixation in the bone is desirable.

The compounds of the invention may be used alone or in combination with other suitable active agents. In an eighth aspect of the invention, there is provided as pharmaceutical composition comprising a compound of formula (I) and an additional active agent selected from: a calcitonin or an analogue or derivative thereof, a steroid hormone, a SERM (Selective Estrogen Receptor Modulator), vitamin D or an analog thereof, a bisphosphonate, an RNKL inhibitor, PTH, a PTH fragment or a PTH derivative, or a cathepsin K inhibitor for simultaneous, separate or sequential use.

Agents of the invention may be prepared by processes described below:

### EXAMPLE 1

### 4-Bromo-2-(4-isopropyl-phenyl)-7-methoxy-1-(2-methoxy-ethyl)-5-(2-methylsulfanyl-pyridin-3-ylmethyl)-1H-benzoimidazole

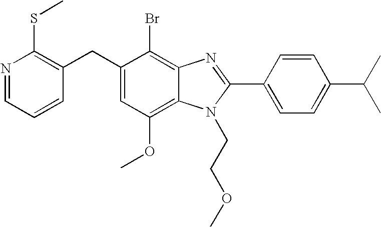

A mixture of 0.95 g (1.97 mmol) 2-(4-isopropyl-phenyl)-7-methoxy-1-(2-methoxy-ethyl)-5-(2-methylsulfanyl-pyridin-3-ylmethyl)-1H-benzoimidazole, 0.103 ml bromine 70 ml acetic acid is stirred at room temperature for 1 h. After that the reaction mixture is poured on water and extracted 3 times with ethyl acetate. The organic layer is washed with 4N NaOH solution (2×), water (3×) and brine (2×), dried (MgSO₄) and concentrated in vacuo. The residue is purified by flash-chromatography on silica gel (hexanes/EtOAc 3:1=>EtOAc) and recrystallisation from diethyl ether/hexane to give the title compound as white crystals.

Rₜ=2.26 min (Waters Symmetry C8, 2.1×50 mm, detection 210-250 nM, 5% to 100% CH₃CN in H₂O in 2 min+0.1% TFA, flow rate 1.0 ml/min)

MS: 540 (M+1)⁺ (⁷⁹Br), 542 (M+1)⁺ (⁸¹Br)

The starting materials can be prepared as follows:

### a) 2-(4-Isopropyl-phenyl)-7-methoxy-1-(2-methoxy-ethyl)-5-(2-methylsulfanyl-pyridin-3-ylmethyl)-1H-benzoimidazole

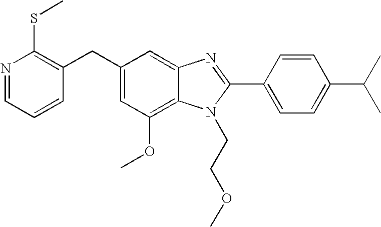

A solution of 10.65 g (14.6 mmol) of [2-(4-isopropyl-phenyl)-7-methoxy-1-(2-methoxy-ethyl)-1H-benzoimidazol-5-yl]-(2-methylsulfanyl-pyridin-3-yl)-methanol in 200 ml formic acid is heated to reflux temperature. Over a period of ca. 24 h, 18.2 g of zinc (powder) is added in small portions at reflux temperature. After that the reaction mixture is cooled to room temperature, poured on water and extracted 3 times with ethyl acetate. The organic layer is washed with 4N NaOH solution (2×), water (3×) and brine (2×), dried (MgSO₄) and concentrated in vacuo. The residue is purified by flash-chromatography on silica gel (hexanes/EtOAc 2:1=>EtOAc) followed by recrystallisation from diethyl ether/hexane to give the title compound as colorless crystals.

### b) [2-(4-Isopropyl-phenyl)-7-methoxy-1-(2-methoxy-ethyl)-1H-benzoimidazol-5-yl]-(2-methylsulfanyl-pyridin-3-yl)-methanol

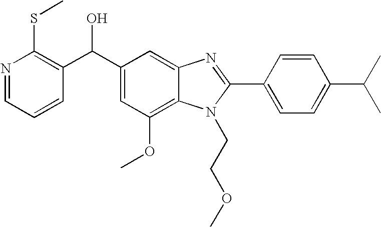

To a solution of 8.86 g (43.4 mmol) 3-bromo-2-methylsulfanyl-pyridine in 165 ml dry THF, n-BuLi (31 ml, 1.6M in hexane) is added slowly at −70° C. Stirring is continued at this temperature for 2 h, and a solution of 10 g (28.4 mmol) 2-(4-isopropyl-phenyl)-7-methoxy-1-(2-methoxy-ethyl)-1H-benzoimidazole-5-carbaldehyde (preparation of this compound is described in WO 2005/068433 A1) in 165 ml dry THF is added within 10 min. The reaction mixture is allowed to reach room temperature and is poured on water and extracted 3 times with ethyl acetate. The organic layer is washed with water (3×) and brine (2×), dried (Mg SO₄) and concentrated in vacuo. The residue is purified by flash-chromatography on silica gel (hexanes/EtOAc 1:1=>EtOAc) to give the title compound as a yellow foam.

### c) 3-Bromo-2-methylsulfanyl-pyridine

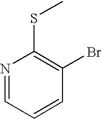

A mixture of 10 g (50.9 mmol) 3-bromo-2-chloro-pyridine, 4.66 g (63.1 mmol) sodium methane-thiolate in 100 ml dry THF is stirred at 60° C. for 7 h. After that the reaction mixture is cooled to room temperature and poured on water and extracted 3 times with ethyl acetate. The organic layer is washed with water (1×) and brine (1×), dried (MgSO₄) and concentrated in vacuo to give the title compound as a colorless oil.

### EXAMPLE 2

### 2-(4-Isopropyl-phenyl)-7-methoxy-1-(2-methoxy-ethyl)-5-(2-methylsulfanyl-pyridin-3-ylmethyl)-4-trifluoromethyl-1H-benzoimidazole

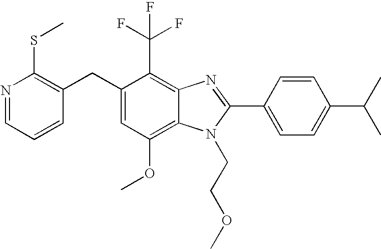

A mixture of 530 mg (0.7 mmol) 4-iodo-2-(4-isopropyl-phenyl)-5-(2-methanesulfinyl-pyridin-3-ylmethyl)-7-methoxy-1-(2-methoxy-ethyl)-1H-benzoimidazole, 62.7 mg (0.351 mmol) copper (I) iodide and 0.225 ml (1.76 mmol) methyl-2,2-difluoro-2-(fluorosulfonyl)acetate (Aldrich 390755) in 15 ml dimethylformamide is stirred at 120° C. for 4 h. After that the reaction mixture is cooled to room temperature, poured on water and extracted 3 times with ethyl acetate. The organic layer is washed with water (3×) and brine (2×), dried (MgSO₄) and concentrated in vacuo. The residue is purified by flash-chromatography on silica gel (hexanes/EtOAc 3:1=>2:1) followed by recrystallisation from diethyl ether/hexane to give the title compound as colorless crystals.

Rₜ=2.38 min (Waters Symmetry C8, 2.1×50 mm, detection 210-250 nM, 5% to 100% CH₃CN in H₂O in 2 min+0.1% TFA, flow rate 1.0 ml/min)

MS: 530 (M+1)⁺

The starting materials can be prepared as follows:

### a) 4-Iodo-2-(4-isopropyl-phenyl)-5-(2-methanesulfinyl-pyridin-3-ylmethyl)-7-methoxy-1-(2-methoxy-ethyl)-1H-benzoimidazole

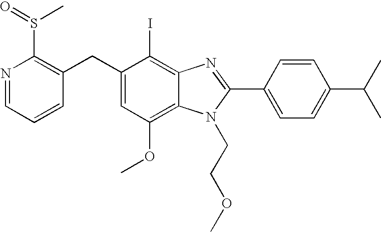

A mixture of 2.38 g (5.0 mmol) 2-(4-isopropyl-phenyl)-7-methoxy-1-(2-methoxy-ethyl)-5-(2-methylsulfanyl-pyridin-3-ylmethyl)-1H-benzoimidazole, 1.3 g iodine and 1.6 g silver sulfate in 50 ml acetic acid is stirred at 80° C. for 4 h, where another 1.3 g iodine and 1.6 g silver sulfate are added (as one equivalent of reagents is used to oxidize the sulfur, addition of another equivalent is necessary). Stirring is continued for 3 h. After that the reaction mixture is cooled to room temperature, poured on water and extracted 3 times with ethyl acetate. The organic layer is washed with 4N NaOH solution, water (3×) and brine (2×), dried (MgSO₄) and concentrated in vacuo. The residue is recrystallised from dichloromethane/diethyl ether to give the title compound as off-white crystals.

### EXAMPLE 3

### 2-(4-Isopropyl-phenyl)-5-(2-methanesulfinyl-pyridin-3-ylmethyl)-7-methoxy-1-(2-methoxy-ethyl)-4-trifluoromethyl-1H-benzoimidazole

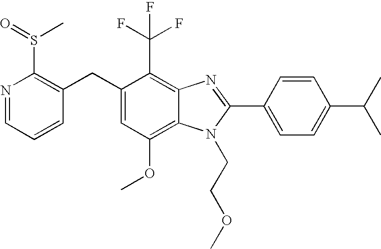

A mixture of 30 mg (0.057 mmol) 2-(4-isopropyl-phenyl)-7-methoxy-1-(2-methoxy-ethyl)-5-(2-methylsulfanyl-pyridin-3-ylmethyl)-4-trifluoromethyl-1H-benzoimidazole (example 2) and 6.4 microliter hydrogen peroxide/water solution in 1 ml acetic acid are stirred at room temperature for 3 h. After that the reaction mixture is diluted with ethyl acetate and washed with 4N NaOH solution (1×), water (1×) and NaHSO₃ solution (1×), dried (MgSO₄) and concentrated in vacuo to give the title compound as a colorless oil.

Rₜ=2.11 min (Waters Symmetry C8, 2.1×50 mm, detection 210-250 nM, 5% to 100% CH₃CN in H₂O in 2 min+0.1% TFA, flow rate 1.0 ml/min)

MS: 546 (M+1)⁺

### EXAMPLE 4

### 2-(4-Isopropyl-phenyl)-5-(2-methanesulfonyl-pyridin-3-ylmethyl)-7-methoxy-1-(2-methoxy-ethyl)-4-trifluoromethyl-1H-benzoimidazole

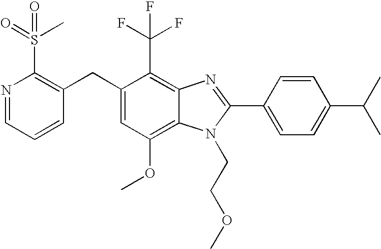

A mixture of 16 mg (0.029 mmol) 2-(4-isopropyl-phenyl)-5-(2-methanesulfinyl-pyridin-3-ylmethyl)-7-methoxy-1-(2-methoxy-ethyl)-4-trifluoromethyl-1H-benzoimidazole (example 3) and 6.0 microliter hydrogen peroxide/water solution in 1 ml acetic acid are stirred at room temperature for 3 h. After that the reaction mixture is diluted with ethyl acetate and washed with 4N NaOH solution (1×), water (1×) and NaHSO3 solution (1×), dried (MgSO₄) and concentrated in vacuo to give the title compound as a colorless oil.

Rₜ=2.27 min (Waters Symmetry C8, 2.1×50 mm, detection 210-250 nM, 5% to 100% CH₃CN in H₂O in 2 min+0.1% TFA, flow rate 1.0 ml/min)

MS: 562 (M+1)⁺

### EXAMPLE 5

### 4-Bromo-2-(4-isopropyl-phenyl)-7-methoxy-1-(2-methoxy-ethyl)-5-(2-methylsulfinyl-pyridin-3-ylmethyl)-1H-benzoimidazole

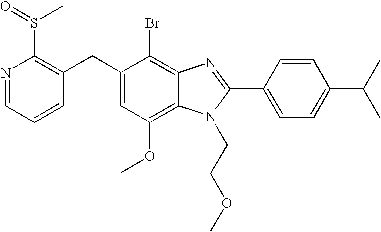

The title compound can be prepared from 4-bromo-2-(4-isopropyl-phenyl)-7-methoxy-1-(2-methoxy-ethyl)-5-(2-methylsulfanyl-pyridin-3-ylmethyl)-1H-benzoimidazole using the same methodology as described for the preparation of example 3.

Rₜ=2.04 min (Waters Symmetry C8, 2.1×50 mm, detection 210-250 nM, 5% to 100% CH₃CN in H₂O in 2 min+0.1% TFA, flow rate 1.0 ml/min)

MS: 556 (M+1)⁺ (⁷⁹Br), 558 (M+1)⁺ (⁸¹Br)

### EXAMPLE 6

### 2-(4-Isopropyl-phenyl)-7-methoxy-1-(2-methoxy-ethyl)-5-(2-methoxy-pyridin-3-ylmethyl)-4-trifluoromethyl-1H-benzoimidazole

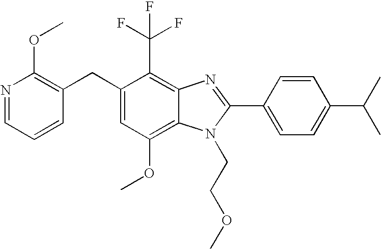

Rₜ=2.11 min (Waters Symmetry C8, 2.1×50 mm, detection 210-250 nM, 5% to 100% CH₃CN in H₂O in 2 min+0.1% TFA, flow rate 1.0 ml/min)

MS: 514 (M+1)⁺

The title compound is prepared using the same methodology as described for the preparation of example 2 from 3-bromo-2-methoxy-pyridine instead of 3-bromo-2-methylsulfanyl-pyridine.

### Alternative Procedure:

Rₜ=2.39 min (Phenomenex Luna C8, 2×50 mm, 3 μm, detection 190-270 nm, Solvent: A: CH₃CN/H₂O/TFA=95/5/0.1, B: CH₃CN/TFA=100/0.1, Gradient: starting with 5% B and coming up to 95% B within 2 min then 95% B for 1 min and going back to 5% B within 0.3 min, flow rate 1.0 ml/min)

A solution of 2-(4-isopropyl-phenyl)-5-(2-methanesulfonyl-pyridin-3-ylmethyl)-7-methoxy-1-(2-methoxy-ethyl)-4-trifluoromethyl-1H-benzoimidazole (100 mg, 0.177 mmol, for preparation see Example 4) in dioxane (2 ml) is treated with sodium methylate (201 mg, 3.54 mmol). A small amount of MeOH (1 ml) needs to be added in order to obtain a solution. The reaction mixture is stirred at 50° C. for 60 hrs. Work-up is done by the addition of water (10 ml) followed by stirring for 2 hrs at room temperature resulting in the formation of white crystals. They are filtered off and washed with water to give pure product.

### EXAMPLE 7

### 5-(2-Ethoxy-pyridin-3-ylmethyl)-2-(4-Isopropyl-phenyl)-7-methoxy-1-(2-methoxy-ethyl)-4-trifluoromethyl-1H-benzoimidazole

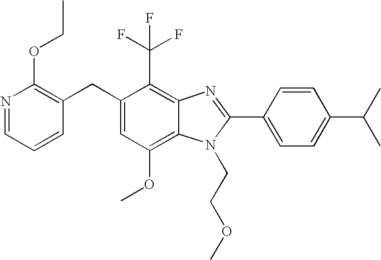

Rₜ=2.45 min (Phenomenex Luna C8, 2×50 mm, 3 μm, detection 190-270 nm, Solvent: A: CH₃CN/H₂O/TFA=95/5/0.1, B: CH₃CN/TFA=100/0.1, Gradient: starting with 5% B and coming up to 95% B within 2 min then 95% B for 1 min and going back to 5% B within 0.3 min, flow rate 1.0 ml/min)

MS: 528 (M+1)⁺

A suspension of 2-(4-isopropyl-phenyl)-5-(2-methanesulfonyl-pyridin-3-ylmethyl)-7-methoxy-1-(2-methoxy-ethyl)-4-trifluoromethyl-1H-benzoimidazole (100 mg, 0.177 mmol, for preparation see Example 4) in dioxane (1.30 ml) is mixed with a solution of sodium ethylate in ethanol (21%, 1.3 ml, 3.5 mmol). The resulting solution is stirred overnight at 50° C. The reaction mixture is then cooled to room temperature, mixed with aqueous NaHCO₃ solution (saturated) and extracted with ethyl acetate (3×). The combined organic layers are washed with water and brine, dried over Na₂SO₄ and the solvent removed under reduced pressure. The crude product is purified by chromatography (silica, solvent: hexane/ethyl acetate 75/25) to yield the product in form of a pale yellow powder.

### EXAMPLE 8

### 5-(2-Isopropoxy-pyridin-3-ylmethyl)-2-(4-Isopropyl-phenyl)-7-methoxy-1-(2-methoxy-ethyl)-4-trifluoromethyl-1H-benzoimidazole

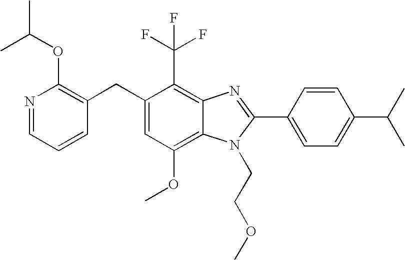

Rₜ=2.50 min (Phenomenex Luna C8, 2×50 mm, 3 μm, detection 190-270 nm, Solvent: A: CH₃CN/H₂O/TFA=95/5/0.1, B: CH₃CN/TFA=100/0.1, Gradient: starting with 5% B and coming up to 95% B within 2 min then 95% B for 1 min and going back to 5% B within 0.3 min, flow rate 1.0 ml/min)

MS: 542.1 (M+1)⁺, 1083.3 (2M+1)⁺

A suspension of 2-(4-isopropyl-phenyl)-5-(2-methanesulfonyl-pyridin-3-ylmethyl)-7-methoxy-1-(2-methoxy-ethyl)-4-trifluoromethyl-1H-benzoimidazole (100 mg, 0.177 mmol, for preparation see Example 4) n dioxane (1.30 ml) is mixed with isopropyl alcohol (208 μl, 3.54 mmol). NaH (60% in mineral oil, 3.9 mmol) is added and the resulting reaction mixture is stirred at 50° C. for several days until more than 90% of conversion to the desired product can be determined by LC/MS analysis. Then, saturated aqueous NaHCO₃ solution (50 ml) is added and the resulting mixture is extracted with ethyl acetate (3×). The combined organic phases are washed with water and brine, dried over Na₂SO₄ and the solvent removed under reduced pressure. The crude product is purified by column chromatography (ethyl acetate/hexanes) to yield pure material as a colorless oil.

### EXAMPLE 9

### 2-(4-Isopropyl-phenyl)-7-methoxy-1-(2-methoxy-ethyl)-5-(2-prop-2-ynyloxy-pyridin-3-ylmethyl)-4-trifluoromethyl-1H-benzoimidazole

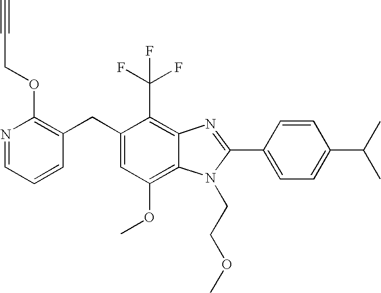

Rₜ=2.44 min (Phenomenex Luna C8, 2×50 mm, 3 μm, detection 190-270 nm, Solvent: A: CH₃CN/H₂O/TFA=95/5/0.1, B: CH₃CN/TFA=100/0.1, Gradient: starting with 5% B and coming up to 95% B within 2 min then 95% B for 1 min and going back to 5% B within 0.3 min, flow rate 1.0 ml/min)

MS: 538.1 (M+1)⁺, 1075.3 (2M+1)⁺

A suspension of 2-(4-isopropyl-phenyl)-5-(2-methanesulfonyl-pyridin-3-ylmethyl)-7-methoxy-1-(2-methoxy-ethyl)-4-trifluoromethyl-1H-benzoimidazole (100 mg, 0.177 mmol, for preparation see Example 4) in dioxane (1.30 ml) is mixed with propargyl alcohol (208 μl, 3.54 mmol). NaH (60% in mineral oil, 156 mg, 3.9 mmol) is added and the resulting solution stirred overnight at 50° C., after which additional NaH (60% in mineral oil, 20 mg) is added. Stirring is continued at 50° C. until LC/MS analysis shows approx. 95% conversion to the desired product (16 hrs). Then water (5 ml) is added to the mixture upon which the product starts to crystallize. The material was filtered off and washed with water to give pure white crystals.

### EXAMPLE 10

### 2-(4-Isopropyl-phenyl)-7-methoxy-5-[2-(2-methoxy-ethoxy)-pyridin-3-ylmethyl]-1-(2-methoxy-ethyl)-4-trifluoromethyl-1H-benzoimidazole

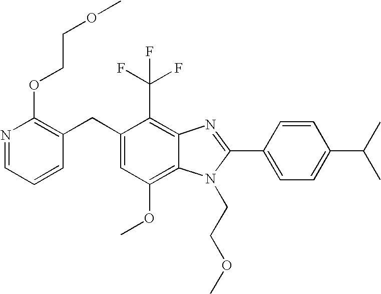

Rₜ=2.18 min (Phenomenex Luna C8, 2×50 mm, 3 μm, detection 190-270 nm, Solvent: A: CH₃CN/H₂O/TFA=95/5/0.1, B: CH₃CN/TFA=100/0.1, Gradient: starting with 5% B and coming up to 95% B within 2 min then 95% B for 1 min and going back to 5% B within 0.3 min, flow rate 1.0 ml/min)

MS: 558 (M+1)⁺

A solution of 2-(4-isopropyl-phenyl)-5-(2-methanesulfonyl-pyridin-3-ylmethyl)-7-methoxy-1-(2-methoxy-ethyl)-4-trifluoromethyl-1H-benzoimidazole (100 mg, 0.177 mmol, for preparation see Example 4) in dioxane (2 ml) is mixed with 2-methoxyethanol (281 μl, 3.56 mmol). NaH (60% in mineral oil, 14.2 mg, 0.36 mmol) is added and the resulting reaction mixture is stirred for 60 hrs at 60° C. The reaction mixture is quenched with saturated aqueous NaHCO₃ solution and extracted with ethyl acetate (3×). The combined organic layers are washed with water and brine, dried over Na₂SO₄ and the solvent removed under reduced pressure. The crude product is purified by chromatography (ethyl acetate/hexanes) to give a pale yellow gluey substance.

### EXAMPLE 11

### (2-{3-[2-(4-Isopropyl-phenyl)-7-methoxy-1-(2-methoxy-ethyl)-4-trifluoromethyl-1H-benzoimidazole-5-ylmethyl]-pyridin-2-yloxy}-ethyl)-dimethylamine

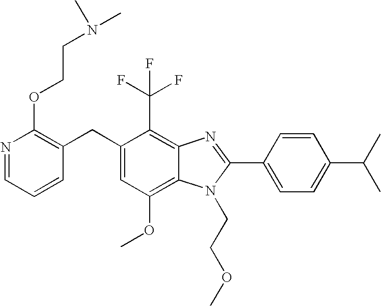

Rₜ=1.87 min (Phenomenex Luna C8, 2×50 mm, 3 μm, detection 190-270 nm, Solvent: A: CH₃CN/H₂O/TFA=95/5/0.1, B: CH₃CN/TFA=100/0.1, Gradient: starting with 5% B and coming up to 95% B within 2 min then 95% B for 1 min and going back to 5% B within 0.3 min, flow rate 1.0 ml/min)

MS: 571 (M+1)⁺

A solution of 2-(4-isopropyl-phenyl)-5-(2-methanesulfonyl-pyridin-3-ylmethyl)-7-methoxy-1-(2-methoxy-ethyl)-4-trifluoromethyl-1H-benzoimidazole (100 mg, 0.177 mmol, for preparation see Example 4) in dioxane (2 ml) is mixed with 2-dimethylaminoethanol (415 μl, 3.56 mmol). NaH (60% in mineral oil, 14.2 mg, 0.36 mmol) is added and the resulting reaction mixture stirred for 6p hrs at 60° C. The reaction mixture is quenched with saturated aqueous NaHCO₃ solution and extracted with ethyl acetate (3×). The combined organic layers are washed with water and brine, dried over Na₂SO₄ and the solvent removed under reduced pressure. The crude product is purified by silicagel chromatography (DCM/MeOH) to give a pale yellow gluey substance.

The Agents of the Invention, as defined above, e.g., of formula (I), particularly as exemplified, in free or pharmaceutically acceptable acid addition salt form, exhibit pharmacological activity and are useful as pharmaceuticals, e.g. for therapy, in the treatment of diseases and conditions as hereinafter set forth.

### Inositol Phosphate Formation Assay:

To determine antagonistic activity at the human parathyroid calcium-sensing receptor (PCaR), compounds are tested in functional assays measuring the inhibition of calcium-induced inositol phosphate formation in CCL39 fibroblasts stably transfected with human PCaR.

Cells are seeded into 24 well plates and grown to confluence. Cultures are then labelled with [³H]inositol (74 Mbq/ml) in serum-free medium for 24 h. After labelling, cells are washed once with a modified Hepes-buffered salt solution (mHBS: 130 mM NaCl, 5.4 mM KCl, 0.5 mM CaCl₂, 0.9 mM MgSO₄, 10 mM glucose, 20 mM HEPES, pH 7.4) and incubated with mHBS at 37° C. in the presence of 20 mM LiCl to block inositol monophosphatase activity. Test compounds are added 3 minutes before stimulating PCaR with 5.5 mM calcium and incubations continued for further 20 min. Thereafter, cells are extracted with 10 mM ice-cold formic acid and inositol phosphates formed are determined using anion exchange chromatography and liquid scintillation counting.

### Assay for Intracellular Free Calcium:

An alternative method to determine antagonism at the PCaR consists in measuring the inhibition of intracellular calcium transients stimulated by extracellular calcium. CCL39 fibroblasts stably transfected with human PCaR are seeded at 40′000 cells/well into 96-well Viewplates and incubated for 24 hours. Medium is then removed and replaced with fresh medium containing 2 μM Fluo-3 AM (Molecular Probes, Leiden, The Netherlands), In routine experiments, cells are incubated at 37° C., 5% CO₂ for 1 h. Afterwards, plates are washed twice with mHBS and wells are refilled with 100 μl mHBS containing the test compounds. Incubation is continued at room temperature for 15 minutes. To record changes of intracellular free calcium, plates are transferred to fluorescence-imaging plate reader (Molecular Devices, Sunnyvale, Calif., USA). A baseline consisting in 5 measurements of 0.4 seconds each (laser excitation 488 nm) is recorded. Cells are then stimulated with calcium (2.5 mM final), and fluorescence changes recorded over a period of 3 minutes.

When measured in the above assays, Agents of the Invention typically have IC₅₀s in the range from about 1000 nM down to about 10 nM or less. To illustrate the activity of the agents of the invention, the following examples are provided based on the above described assay:

| Example no. | IC₅₀ [nM] |
|---|---|
| 1 | 3.4 |
| 3 | 2.6 |
| 8 | 3.2 |
| 9 | 1.8 |

It is now well established that controlled treatment of patients with parathyroid hormone (PTH) and analogues and fragments thereof can have a pronounced anabolic effect on bone formation. Thus compounds which promote PTH release, such as the Agents of the Invention may be used for preventing or treating conditions of bone which are associated with increased calcium depletion or resorption or in which stimulation of bone formation and calcium fixation in the bone is desirable.

Agents of the Invention are accordingly indicated for preventing or treating all bone conditions which are associated with increased calcium depletion or resorption or in which stimulation of bone formation and calcium fixation in the bone is desirable, e.g. osteoporosis of various genesis (e.g. juvenile, menopausal, post-menopausal, post-traumatic, caused by old age or by cortico-steroid therapy or inactivity), fractures, osteopathy, including acute and chronic states associated with skeletal demineralisation, osteo-malacia, periodontal bone loss or bone loss due to arthritis or osteoarthritis or for treating hypoparathyroidism.

Further diseases and disorders which might be prevented or treated include e.g. seizures, stroke, head trauma, spinal cord injury, hypoxia-induced nerve cell damage such as in cardiac arrest or neonatal distress, epilepsy, neurodegenerative diseases such as Alzheimer's disease, Huntington's disease and Parkinson's disease, dementia, muscle tension, depression, anxiety, panic disorder, obsessive-compulsive disorder, post-traumatic stress disorder, schizophrenia, neuroleptic malignant syndrome, congestive heart failure; hypertension; gut motility disorders such as diarrhea, and spastic colon and dermatological disorders, e.g. in tissue healing, for example burns, ulcerations and wounds.

The Agents of the Invention are particularly indicated for preventing or treating osteoporosis of various genesis.

For all the above uses, an indicated daily dosage is in the range from about 0.03 to about 1000 mg, preferably 0.03 to 200 mg, more preferably 0.03 to 30, yet more preferably 0.1 to 10 mg of a compound of the invention. Agents of the Invention may be administered twice a day or up to twice a week.

The Agents of the Invention may be administered in free form or in pharmaceutically acceptable salt form. Such salts may be prepared in conventional manner and exhibit the same order of activity as the free compounds. The present invention also provides a pharmaceutical composition comprising an Agent of the Invention in free base form or in pharmaceutically acceptable salt form in association with a pharmaceutically acceptable diluent or carrier. Such compositions may be formulated in conventional manner. The Agents of the Invention may be administered by any conventional route, for example parenterally e.g. in the form of injectable solutions or suspensions, enterally, e.g. orally, for example in the form of tablets or capsules or in a transdermal, nasal or a suppository form.

In accordance with the foregoing the present invention further provides:

a) an Agent of the Invention or a pharmaceutically acceptable salt thereof for use as a pharmaceutical;
b) a method for preventing or treating above mentioned disorders and diseases in a subject in need of such treatment, which method comprises administering to said subject an effective amount of an Agent of the Invention or a pharmaceutically acceptable salt thereof;
c) an Agent of the Invention or a pharmaceutically acceptable salt thereof for use in the preparation of a pharmaceutical composition e.g. for use in the method as in b) above.

According to a further embodiment of the invention, the Agents of the Invention may be employed as adjunct or adjuvant to other therapy, e.g. a therapy using a bone resorption inhibitor or a bone formation promoter, for example as in osteoporosis therapy or in cancer therapy, in particular a therapy employing calcium, a calcitonin or an analogue or derivative thereof, e.g. salmon, eel or human calcitonin, a steroid hormone, e.g. an estrogen, a partial estrogen agonist or estrogen-gestagen combination, a SERM (Selective Estrogen Receptor Modulator) e.g. raloxifene, lasofoxifene, bazedoxifene, arzoxifene, TSE-424, FC1271, Tibolone (Livial®), vitamin D or an analog thereof, a bisphosphonate, e.g. an injectable like zoledronic acid or ibandronate, an RNKL inhibitor, e.g. denosumab, PTH, a PTH fragment or a PTH derivative e.g. PTH (1-84), PTH (1-34), PTH (1-36), PTH (1-38), PTH (1-31)NH₂ or PTS 893, or a cathepsin K inhibitor, e.g. balicatib.

When the Agents of the Invention are administered in conjunction with, e.g. as an adjuvant to bone resorption inhibition therapy, dosages for the co-administered inhibitor will of course vary depending on the type of inhibitor drug employed, e.g. whether it is a steroid or a calcitonin, on the condition to be treated, whether it is a curative or preventive therapy, on the regimen and so forth. Administration may be by any convenient route, e.g. parenterally, orally and may be administered simultaneously, separately or sequentially or at differently timed intervals.

1. A compound of formula (I) or a pharmaceutically acceptable salt or prodrug ester thereof: 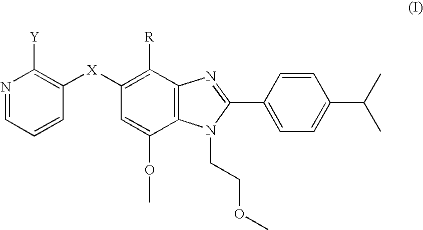
wherein
R is halo or optionally substituted C₁-C₆ alkyl;
X is selected from the group consisting of O, NH, CH₂, CO, SO, SO₂ or S;
Y represents a group selected from the following: optionally substituted C₁-C₆ alkyl, —SR₁, —S(O)R₁, —S(O)₂R₁, —OR₂, wherein R₁ and R₂ are selected from optionally substituted: C₁-C₄ alkyl, C₁-C₄ alkenyl or C₁-C₄ alkynyl;
the optional substituent or substituents on R, R₁, R₂ and Y being independently selected from the group consisting of halogen, hydroxy, C₁-C₆ alkyl, mono or di-C₁-C₆ alkylamino, aminocarbonyl, sulfinyl, sulfonyl, sulfanyl, mono or di-C₁-C₆ alkylaminocarbonyl, amino, carboxy, C₁-C₆ alkoxy, C₂-C₆ alkenyloxy, C₂-C₆ alkynyloxy, C₃-C₁₂ cycloalkyl, C₃-C₁₈ heterocycloalkyl, C₁-C₆ alkylcarbonyl, C₁-C₆ alkoxycarbonyl, nitryl, aryl; all of which, except halogen, are independently optionally substituted by one or more substituents, selected from the group consisting of halogen, hydroxy, C₁-C₆ alkyl, mono or di-C₁-C₆ alkylamino, aminocarbonyl, sulfinyl, sulfonyl, sulfanyl, mono or di-C₁-C₆ alkylaminocarbonyl, amino, carboxy, C₁-C₆ alkoxy, C₃-C₁₂ cycloalkyl, C₃-C₁₈ heterocycloalkyl, C₁-C₆ alkylcarbonyl, C₁-C₆ alkoxycarbonyl, nitryl, aryl.

2. A compound of formula (I′) or a pharmaceutically acceptable salt, or prodrug ester thereof: 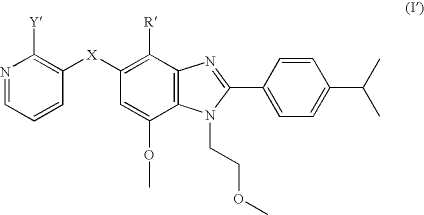
wherein
R′ is halo or optionally substituted C₁-C₆ alkyl;
Y′ represents a group selected from the following: C₁-C₆ alkyl, —SR₁, —S(O)R₁, —S(O)₂R₁, —OR₂, wherein R₁ and R₂ are selected from optionally substituted: C₁-C₄ alkyl, C₁-C₄ alkenyl or C₁-C₄ alkynyl;
the optional substituent or substituents on R, R₁ and R₂ are independently selected from the group consisting of halogen, hydroxy, C₁-C₆ alkyl, mono or di-C₁-C₆ alkylamino, aminocarbonyl, sulfinyl, sulfonyl, sulfanyl, mono or di-C₁-C₆ alkylaminocarbonyl, amino, carboxy, C₁-C₆ alkoxy, C₃-C₁₂ cycloalkyl, C₃-C₁₈ heterocycloalkyl, C₁-C₆ alkylcarbonyl, C₁-C₆ alkoxycarbonyl, nitryl, aryl; all of which, except halogen, are independently optionally substituted by one or more substituents, selected from the group consisting of halogen, hydroxy, C₁-C₆ alkyl, mono or di-C₁-C₆ alkylamino, aminocarbonyl, sulfinyl, sulfonyl, sulfanyl, mono or di-C₁-C₆ alkylaminocarbonyl, amino, carboxy, C₁-C₆ alkoxy, C₃-C₁₂ cycloalkyl, C₃-C₁₈ heterocycloalkyl, C₁-C₆ alkylcarbonyl, C₁-C₆ alkoxycarbonyl, nitryl, aryl.

3. A compound according to claim 1 wherein X is CH₂ or O.

4. A compound according to claim 1 wherein Y is selected from —SR₁, —S(O)R₁, —S(O)₂R₁ and —OR₂,

4. A compound according to claim 1 wherein Y is selected from —SR₁, —S(O)R₁, —S(O)₂R₁ and —OR₂ and R₁ or R₂ is methyl.

5. A compound according to claim 1 wherein R is halo or trifluoromethyl.

6. A compound according to claim 1, selected from:
4-Bromo-2-(4-isopropyl-phenyl)-7-methoxy-1-(2-methoxy-ethyl)-5-(2-methylsulfanyl-pyridin-3-ylmethyl)-1H-benzoimidazole,
2-(4-Isopropyl-phenyl)-7-methoxy-1-(2-methoxy-ethyl)-5-(2-methylsulfanyl-pyridin-3-ylmethyl)-4-trifluoromethyl-1H-benzoimidazole,
4-Bromo-2-(4-isopropyl-phenyl)-5-(2-methanesulfinyl-pyridin-3-ylmethyl)-7-methoxy-1-(2-methoxy-ethyl)-1H-benzoimidazole,
2-(4-Isopropyl-phenyl)-5-(2-methanesulfinyl-pyridin-3-ylmethyl)-7-methoxy-1-(2-methoxy-ethyl)-4-trifluoromethyl-1H-benzoimidazole,
2-(4-Isopropyl-phenyl)-5-(2-methanesulfonyl-pyridin-3-ylmethyl)-7-methoxy-1-(2-methoxy-ethyl)-4-trifluoromethyl-1H-benzoimidazole,
2-(4-Isopropyl-phenyl)-7-methoxy-1-(2-methoxy-ethyl)-5-(2-methoxy-pyridin-3-ylmethyl)-4-trifluoromethyl-1H-benzoimidazole,
5-(2-Ethoxy-pyridin-3-ylmethyl)-2-(4-Isopropyl-phenyl)-7-methoxy-1-(2-methoxy-ethyl)-4-trifluoromethyl-1H-benzoimidazole,
5-(2-Isopropoxy-pyridin-3-yl-methyl)-2-(4-Isopropyl-phenyl)-7-methoxy-1-(2-methoxy-ethyl)-4-trifluoromethyl-1H-benzoimidazole,
2-(4-Isopropyl-phenyl)-7-methoxy-1-(2-methoxy-ethyl)-5-(2-prop-2-ynyloxy-pyridin-3-ylmethyl)-4-trifluoromethyl-1H-benzoimidazole,
2-(4-Isopropyl-phenyl)-7-methoxy-5-[2-(2-methoxy-ethoxy)-pyridin-3-ylmethyl]-1-(2-methoxy-ethyl)-4-trifluoromethyl-1H-benzoimidazole, and
(2-{3-[2-(4-Isopropyl-phenyl)-7-methoxy-1-(2-methoxy-ethyl)-4-trifluoromethyl-1H-benzoimidazole-5-ylmethyl]-pyridin-2-yloxy}-ethyl)-dimethylamine.

7. A pharmaceutical composition comprising a compound of formula (I) as defined in claim 1 in association with a pharmaceutically acceptable excipient, diluent or carrier.

8. A pharmaceutical composition according to claim 7 containing 0.03 to 300 mg of the compound of formula (I).

9. A compound of formula (I) as defined in claim 1 for promoting the release of parathyroid hormone.

10. A method for preventing or treating bone conditions which are associated with increased calcium depletion or resorption or in which stimulation of bone formation and calcium fixation in the bone is desirable in which an effective amount of a compound of formula (I) as defined in claim 1, or a pharmaceutically-acceptable and -cleavable ester, or acid addition salt thereof is administered to a patient in need of such treatment.

11. A process for the preparation of a compound of formula (I) in free or salt form as defined in claim 1, comprising:
(a) for compounds of formula (I) wherein R is optionally substituted C₁-C₆ alkyl, introducing the optionally substituted C₁-C₆ alkyl by reaction of a compound of formula (XV) with a suitable organometallic reagent: 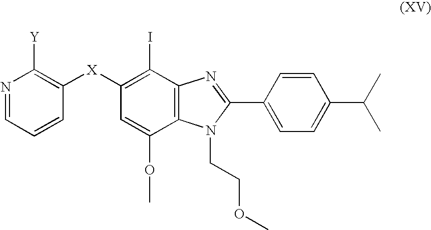
(b) for compounds of formula (I) wherein R is halo, halogenation of a compound of formula (X) using a suitable halogenating agent: 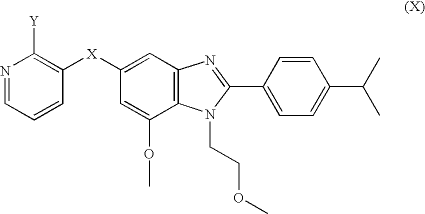
(c) for compounds of formula (I) wherein Y is —SR₁, reduction of a compound of formula (XI) using a suitable reducing agent: 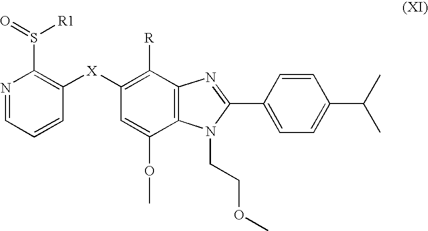
(d) for compounds wherein Y is —S(O)R₁ or —S(O)₂R₁, by oxidation of a compound of formula (XII): 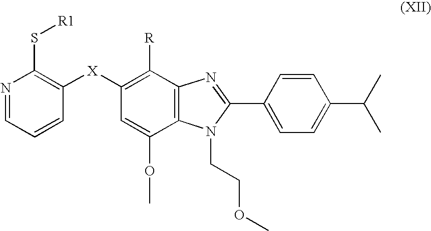
(e) for compounds wherein Y is —OR₂, or —SR₁ by ipso-substitution in the pyridine ring of a compound of formula (XIII): 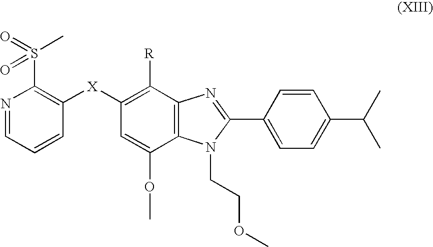
using a suitable nucleophiles such as R₂O⁻ or R₁S⁻.

12. Use of a compound of formula (I) in the manufacture of a medicament for preventing or treating bone conditions which are associated with increased calcium depletion or resorption or in which stimulation of bone formation and calcium fixation in the bone is desirable.

13. A pharmaceutical composition comprising a compound of formula (I) and an additional active agent selected from: a calcitonin or an analogue or derivative thereof, a steroid hormone, a SERM (Selective Estrogen Receptor Modulator), vitamin D or an analog thereof, a bisphosphonate, an RNKL inhibitor, PTH, a PTH fragment or a PTH derivative, or a cathepsin K inhibitor for simultaneous, separate or sequential use.
